(19) United States
(12) Patent Application Publication
(10) Pub. No.: US 2005/0025767 A1
(43) Pub. Date: Feb. 3, 2005

(54) Therapeutic agents for multiple sclerosis

(75) Inventor: Jun Nishihira, Hokkaido (JP)

Correspondence Address: FOLEY AND LARDNER;SUITE 500, 3000 K STREET NW, WASHINGTON, DC, 20007, US

(73) Assignee: Jun NISHIHIRA

(21) Appl. No.: 10/854,787

(22) Filed: May 27, 2004

Related U.S. Application Data
(63) Continuation of application No. PCT/JP02/05368, filed on May 31, 2002.

(30) Foreign Application Priority Data
Nov. 30, 2001 (JP) 366267/2001

(51) Int. Cl. A61K 39/395

(52) U.S. Cl. 424/145.1

(57) ABSTRACT

The present invention provides therapeutic agents for multiple sclerosis containing an antibody binding to macrophage migration inhibitory factor (MIF) as an active ingredient, and the therapeutic agents for multiple sclerosis of the present invention are effective for the conventional form of multiple sclerosis (C-MS) or the optic-spinal form of multiple sclerosis (OpS-Ms).

This application is a continuation-in-part of international application No. PCT/JP02/05368, filed on May 31, 2002.

### TECHNICAL FIELD

The present invention relates to therapeutic agents for multiple sclerosis, more specifically therapeutic agents for multiple sclerosis containing an antibody against macrophage migration inhibitory factor (hereinafter sometimes referred to as MIF).

### BACKGROUND ART

Multiple sclerosis (hereinafter sometimes referred to as MS) is an immune-mediated inflammatory disease that causes multiple lesions in the central nervous system (CNS) such as in the brain and spinal cord to induce various neurological symptoms (blurred vision, dyskinesia, decreased sensation, abnormal sensation, pain, loss of balance/tremor, urinary problems, sexual dysfunction, fatigue, cognitive/emotional disorder, etc.). MS has not been etiologically explained, but it is considered as one of “autoimmune diseases” in which the “immune” system accidentally attacks its own tissue. This disease is thought to be caused by a process in which T-cells and macrophages infiltrate into the white matter to incorrectly recognize the body's “own myelin” covering the axons of neurons in the brain or spinal cord as a foreign enemy and attack it, resulting in inflammation of myelin and then demyelination (destruction of myelin) (Miller S D et al., Immunol Rev (1996) 144:225-244).

MS therapy falls into the following three categories: inflammation relief during the acute phase, prevention of relapse or progression, and symptom relief. During the acute phase, adrenocortical steroids are used to reduce inflammation in demyelinated regions. Interferon P and immunosuppressants are thought to be effective for preventing relapse or progression. In addition, transforming growth factor (TGF)β, which possesses suppressive activity against T-cell proliferation, has been reported to inhibit experimental allergic encephalomyelitis (EAE) (Racke M K et al., J Immunol (1991) 146: 3012-3017). Although immunologically-based treatments have been extensively investigated based on the pathogenic mechanisms of MS, effective and satisfactory therapeutic methods are not well established. Many MS patients alternate between relapse (inflammation in the brain or spinal cord) and remission (recovery from relapse), but are difficult to completely cure, and it is desirable to develop novel and effective therapeutic agents.

Macrophage migration inhibitory factor (MIF) plays a pivotal role in systemic as well as local inflammatory and immune response (Bucala R., FASEB J (1996) 7:19-24; Nishihira J., J Interferon Cytokine Res, (2000) 20:751-762). MIF is a soluble factor secreted from active lymphocytes and identified as a T-cell-derived lymphokine that inhibits the random migration of macrophages in inflammatory loci. Recently, MIF was shown to be homologous to glutathione S-transferase (GST) and to have a detoxifying effect, and it was further reported to have a very wide variety of functions in not only the immune system, but also the endocrine system and cell differentiation/growth; e.g. it is secreted from the anterior pituitary during endotoxin shock and it is induced by low-concentrations of glucocorticoid to act in antagonism to the immunosuppressive effect.

Interestingly, MIF was found as an anterior pituitary-derived hormone, potentiating lethal endotoxemia, and an anti-MIF antibody provided protection from septic shock in mice (Bernhagen J et al., Nature (1993) 365:756-759). MIF, a pluripotent cytokine, exerts a variety of functions, including macrophage activation (enhancement of adherence, phagocytosis and tumoricidal activity) (Nathan C F et al., J Exp Med (1973) 137:275-288; Churchill W H et al., J Immunol (1975) 115: 81-786). More importantly, MIF protein is essential for T-cell activation, and is expressed in a variety of cells, most abundantly in the CNS (Bacher M et al., Proc Natl Aced Sci USA (1996) 93:7849-7854). Anti-MIF antibodies were reported to be useful for treating cytokine-mediated diseases such as shock, inflammation and autoimmune diseases (WO94/26307), but neither description of their relation with MS nor report on the applicability to MS therapy has existed so far.

### DISCLOSURE OF THE INVENTION

An object of the present invention is to provide a novel therapeutic agent for MS.

We accomplished the present invention on the basis of the finding that the above object can be attained by antibodies binding to MIF.

Accordingly, the present invention provides a therapeutic agent for multiple sclerosis containing an antibody binding to macrophage migration inhibitory factor (MIF) as an active ingredient.

The present invention also provides a therapeutic agent for multiple sclerosis containing an antibody binding to macrophage migration inhibitory factor (MIF) to inhibit the binding of macrophage migration inhibitory factor (MIF) to its receptors as an active ingredient.

The present invention also provides said therapeutic agents for multiple sclerosis wherein the antibody is a humanized antibody or chimeric antibody.

The present invention also provides said therapeutic agents for multiple sclerosis wherein the antibody is a monoclonal antibody.

The present invention also provides said therapeutic agents for multiple sclerosis wherein multiple sclerosis is the conventional form of multiple sclerosis (C-MS) or the optic-spinal form of multiple sclerosis (OpS-Ms).

### BRIEF EXPLANATION OF THE DRAWINGS

FIG. 1 shows in situ hybridization of an autopsy specimen obtained from a medulla lesion of an MS patient. Demyelination and perivascular leukocyte infiltration are observed in areas indicated by arrows.

FIG. 2 shows the potential action of anti-MIF antibody for amelioration of neurological damage in MS.

FIG. 3 shows scores of pathological tissues in mice treated with an anti-MIF antibody as compared with control mice.

FIG. 4 shows scores of clinical symptoms in mice treated with an anti-MIF antibody as compared with control mice. Open squares represent the control mice and open diamonds represent the mice treated with the anti-MIF antibody.

### THE MOST PREFERRED EMBODIMENTS OF THE INVENTION

The anti-MIF antibody constituting an active ingredient of therapeutic agents for multiple sclerosis of the present invention may be of any origin, type (monoclonal, polyclonal) and form so far as it is therapeutically effective in the therapeutic agents for MS.

The nucleotide sequence and amino acid sequence of MIF are known and anti-MIF antibodies can be readily prepared by those skilled in the art (Weishui Y. Weiser et al., Proc. Natl. Acad. Sci. USA., Vol. 86 pp. 7522-7526, (1989), U.S. Pat. No. 6,030,615, International Publication WO98/17314, International Publication WO01/64749 and International Publication WO94/26307).

The anti-MIF antibody used in the present invention is not specifically limited so far as it binds to MIF, and can be obtained as a polyclonal or monoclonal antibody using known means. Especially preferred antibodies used in the present invention are monoclonal antibodies from mammals. Monoclonal antibodies from mammals include those produced by hybridomas and those produced by hosts transformed with an expression vector containing the antibody gene by genetic engineering techniques.

Hybridomas producing monoclonal antibodies can be basically constructed by known techniques as follows. MIF is used as an immunizing antigen to immunize host cells according to a standard immunization technique, and the resulting immunized cells are fused to known parent cells by a standard cell fusion technique, and then the fused cells are screened for monoclonal antibody-producing cells by a standard screening method. Specifically, monoclonal antibodies can be prepared as follows.

The gene sequence encoding MIF is inserted into a known expression vector system to transform suitable host cells, and then a desired MIF protein is purified from the host cells or culture supernatants by a known method.

Then, this MIF protein is used as an immunizing antigen. Alternatively, a partial peptide of MIF can also be used as an immunizing antigen. Such a partial peptide can be chemically synthesized from the amino acid sequence of MIF.

The epitope on the MIF molecule recognized by anti-MIF antibodies of the present invention is not specifically limited, but any epitope present on the MIF molecule may be recognized. Thus, any fragment containing an epitope present on the MIF molecule can be used as an antigen for preparing an anti-MIF antibody of the present invention.

Mammals immunized with the immunizing antigen are not specifically limited, but preferably selected considering the compatibility with parent cells used for cell fusion, and rodents such as mice, rats and hamsters are typically used.

Animals are immunized with the immunizing antigen according to known methods. For example, a typical method is intraperitoneal or subcutaneous injection of an immunizing antigen into a mammal. Specifically, an immunizing antigen is diluted or suspended to an appropriate volume in PBS (Phosphate-Buffered Saline) or physiological saline and, if desired, mixed with an appropriate amount of a conventional adjuvant such as Freund's complete adjuvant, and emulsified and then administered to a mammal several times every 4-21 days. An appropriate carrier can be used during immunization with the immunizing antigen.

After immunizing the mammal in this manner and confirming an increase in the serum level of a desired antibody, immunized cells, preferably spleen cells, are collected from the mammal and used for cell fusion.

Myeloma cells from mammals are used as parent cells to which the immunized cells are fused. Suitable myeloma cells include those derived from various known cell lines such as P3 (P3x63Ag8.653) (J. Immnol. (1979) 123, 1548-1550), P3x63Ag8U.1 (Current Topics in Microbiology and Immunology (1978) 81, 1-7), NS-1 (Kohler. G. and Milstein, C. Eur. J. Immunol. (1976) 6, 511-519), MPC-11 (Margulies. D. H. et al., Cell (1976) 8, 405-415), SP2/0 (Shulman, M. et al., Nature (1978) 276, 269-270), FO (de St. Groth, S. F. et al., J. Immunol. Methods (1980) 35, 1-21), S194 (Trowbridge, I. S. J. Exp. Med. (1978) 148, 313-323) and R210 (Galfre, G. et al., Nature (1979) 277, 131-133).

Cell fusion of the immunized cells to myeloma cells can be basically performed according to known methods, such as the method of Kohler and Milstein et al. (Kohler. G. and Milstein, C., Methods Enzymol. (1981) 73: 3-46).

More specifically, cell fusion is performed in a conventional nutrient culture medium in the presence of, e.g., a cell fusion promoter such as polyethylene glycol (PEG) or Sendai virus (HVJ) and, if desired, an additive for improving the fusion efficiency such as dimethyl sulfoxide.

Immunized cells and myeloma cells can be used in any ratio. For example, the ratio of immunized cells to myeloma cells is preferably 1-10. Suitable culture media for the cell fusion include, for example, RPMI1640 and MEM well-suitable for culturing the myeloma cell lines mentioned above and other conventional culture media used for this type of cell culture, optionally in combination with serum supplements such as fetal calf serum (FCS).

Cell fusion is performed by thoroughly mixing given amounts of the immunized cells and myeloma cells in the culture medium, adding a PEG solution (e.g. with an average molecular weight of about 1000-6000) preheated at about 37° C. normally at a concentration of 30-60% (w/v) to the mixture and mixing the cell solution to form desired fused cells (hybridomas). Subsequently, cell fusion promoters or the like undesirable for the growth of hybridomas are removed by repeating the steps of gradually adding a suitable culture medium and centrifuging the mixture to remove supernatants.

Thus obtained hybridomas are selected by incubation in a conventional selective culture medium such as a HAT (a culture medium comprising hypoxanthine, aminopterin and thymidine). The incubation in the HAT medium is continued for a sufficient period to kill cells other than desired hybridomas (non-fused cells); typically, several days to several weeks. Then, hybridomas producing a desired antibody are screened by conventional limiting dilution and single copies are cloned.

As an alternative method to obtaining hybridomas by immunizing non-human animals with an antigen as above, desired human antibodies having a binding activity for MIF can be obtained by in vitro immunizing human lymphocytes with MIF and fusing the immunized lymphocytes to permanently divide human myeloma cells (see JPB HEI 1-59878). Human antibodies against MIF can also be obtained from anti-MIF antibody-producing cells, which themselves are obtained by administering MIF as antigen to a transgenic animal having all human antibody gene repertoires, and then immortalized (see International Publications Nos. WO 94/25585, WO 93/12227, WO 92/03918 and WO 94/02602).

Hybridomas producing monoclonal antibodies prepared in this manner can be subcultured in conventional culture media and stored for a long period in liquid nitrogen.

Monoclonal antibodies can be obtained from said hybridomas as culture supernatants by culturing said hybridomas according to conventional methods or as ascites by growing said hybridomas in a mammal compatible with them. The former method is suitable for obtaining high-purity antibodies while the latter method is suitable for mass production of antibodies.

In the present invention, recombinant monoclonal antibodies can be used, which are produced by transforming a host with a suitable vector containing an antibody gene cloned from a hybridoma using genetic engineering techniques (e.g. see Vandamme, A. M. et al., Eur. J. Biochem. (1990) 192, 767-775, 1990).

Specifically, the mRNA sequences encoding the variable regions (V regions) of an anti-MIF antibody are isolated from hybridomas producing the anti-MIF antibody. The mRNA is isolated by known methods such as guanidine ultracentrifugation (Chirgwin, J. M. et al., Biochemistry (1979) 18, 5294-5299) and AGPC (Chomczynski, P. et al., Anal. Biochem. (1987) 162, 156-159) to prepare total RNA, from which a desired mRNA is prepared using an mRNA Purification Kit (Pharmacia) or other means. The mRNA can also be directly prepared by using a QuickPrep mRNA Purification Kit (Pharmacia).

The cDNA sequences for the V regions are synthesized from the mRNA obtained above using a reverse transcriptase. The cDNA is synthesized using an AMV Reverse Transcriptase First-strand cDNA Synthesis Kit (Seikagaku) or the like. The cDNA can be synthesized and amplified by 5′-RACE (Frohman, M. A. et al., Proc. Natl. Acad. Sci. USA (1988)85, 8998-9002, Belyavsky, A. et al., Nucleic Acids Res. (1989)17, 2919-2932) using a 5′-Ampli FINDER RACE Kit (Clontech) and PCR or the like.

A desired DNA fragment is purified from the resulting PCR product and fused to a vector DNA. Then, a recombinant vector is prepared from the fused system and transferred into E. coli or the like and colonies are selected to prepare a desired recombinant vector. Then, the nucleotide sequence of a desired DNA is confirmed by known methods such as dideoxynucleotide chain termination.

After the DNA sequences encoding the V regions of the desired anti-MIF antibody have been obtained, they are integrated into an expression vector containing the DNA sequences encoding the constant regions (C regions) of the desired antibody.

To prepare an anti-MIF antibody used in the present invention, the gene for the antibody is integrated into an expression vector in such a manner that it can be expressed under the control of regulatory regions such as enhancers and promoters. Then, a host cell is transformed with this expression vector to express the antibody.

The gene for the antibody can be expressed by separately integrating the DNA encoding the heavy chain (H chain) or light chain (L chain) of the antibody to simultaneously transform a host cell, or integrating the DNA encoding the heavy and light chains into a single expression vector to transform a host cell (see WO 94/11523).

Not only the host cells described above but also transgenic animals can be used to produce recombinant antibodies. For example, an antibody gene is inserted into a gene encoding a protein produced specifically in milk (such as goat β casein) to prepare a fusion gene. A DNA fragment containing the fusion gene bearing the antibody gene is injected into the embryo of a goat and this embryo is implanted into a female goat. A desired antibody is obtained from the milk produced by transgenic goats born from the goat impregnated with the embryo or progeny thereof. To increase the amount of the antibody-containing milk produced by the transgenic goats, hormones may be administered to the transgenic goats as appropriate (Ebert, K. M. et al., Bio/Technology (1994) 12, 699-702).

In the present invention, recombinant antibodies, i.e. antibodies artificially modified to reduce antigenicity in humans or to attain other purposes, such as chimeric antibodies and humanized antibodies can be used. These modified antibodies can be prepared by known processes.

Chimeric antibodies can be obtained by linking the DNA sequences encoding the variable regions of the antibody obtained as above to the DNA sequences encoding the constant regions of the human antibody and transforming a host with an expression vector containing the linked sequences to allow it to produce a chimeric antibody. This known method can be used to obtain chimeric antibodies useful for the present invention.

Humanized antibodies are also called reshaped human antibodies and are obtained by grafting the complementarity-determining regions (CDRs) of an antibody from a non-human mammal such as a mouse into the complementarity-determining regions of a human antibody, and typical gene recombination techniques for preparing them are also known (see European Patent Publication EP 125023, WO 96/02576).

Specifically, DNA sequences designed to link the CDRs of a mouse antibody to the framework regions (FRs) of a human antibody are synthesized by PCR using several oligonucleotides prepared to have terminal overlapping regions of both CDRs and FRs as primers (see the method described in WO 98/13388).

The framework regions of the human antibody linked by the CDRs are selected in such a manner that the complementarity-determining regions form an appropriate antigen-binding site. If necessary, reshaped humanized antibodies may have some amino acid changes in the framework regions of the variable regions so that the complementarity-determining regions form an appropriate antigen-binding site (Sato, K. et al., Cancer Res. (1993) 53, 851-856).

The constant regions that can be used in chimeric antibodies and humanized antibodies consist of those of a human antibody, e.g. Cγ1, Cγ2, Cγ3 and Cγ4 in the heavy chain and Cκ and Cλ in the light chain. The constant regions of the human antibody can be modified to improve the stability of the antibody or production thereof.

Chimeric antibodies consist of the variable regions of an antibody from a non-human mammal such as a mouse and the constant regions of a human antibody. On the other hand, humanized antibodies consist of the complementarity-determining regions of an antibody from a non-human mammal and the framework regions and constant regions of a human antibody. Humanized antibodies are useful as active ingredients of therapeutic agents of the present invention because of the reduced antigenicity in humans.

Antibodies used in the present invention are not limited to whole molecule antibodies but may be antibody fragments or modified fragments and include divalent and monovalent antibodies so far as they bind to MIF. For example, antibody fragments include Fab, (Fab′)₂, Fv, Fab/c having one Fab and a whole Fc, or single chain Fv (scFv) in which the heavy and light chain Fv fragments are joined with a suitable linker. Specifically, an antibody is treated with an enzyme such as papain or pepsin to produce antibody fragments, or the genes encoding these antibody fragments are constructed and introduced into an expression vector and then expressed in a suitable host cell (for example, see Co, M. S. et al., J. Imunol. (1994) 152, 2968-2976, Better, M. & Horwitz, A. H. Methods in Enzymology (1989) 178, 476-496, Academic Press, Inc., Plueckthun, A. & Skerra, A. Methods in Enzymology (1989) 178, 476-496, Academic Press, Inc., Lamoyi, E, Methods in Enzymology (1989) 121, 652-663, Rousseaux, J. et al, Methods in Enzymology (1989) 121, 663-669, Bird, R. E. et al., TIBTECH (1991) 9, 132-137).

Fragments scFv are obtained by joining the heavy chain variable region and the light chain variable region of an antibody. In the scFv fragments, the heavy chain variable region and the light chain variable region are joined by a linker, preferably a peptide linker (Huston, J. S. et al., Proc. Natl. Acad. Sci. U.S.A. (1988) 85, 5879-5883). The heavy chain variable region and the light chain variable region in the scFv fragments may be derived from any antibody described herein. The peptide linker used for joining the variable regions is, e.g., a single-stranded peptide consisting of 12-19 amino acid residues.

The DNA sequences encoding the scFv fragments are obtained by PCR amplification using, as templates, the entire sequences of the DNA encoding the heavy chain or the heavy chain variable region, and the DNA encoding the light chain or the light chain variable region of the antibody or a part thereof encoding a desired amino acid sequence, in combination with primer pairs defining both ends of these sequences, and then using the DNA encoding a peptide linker region in combination with a primer pair defining both ends thereof to be linked to the heavy and light chains.

Once the DNA sequences encoding the scFv fragments are prepared, an expression vector containing these fragments and a host transformed with the expression vector can be obtained by conventional methods, and the host can be used to give scFv by conventional methods.

These antibody fragments can be produced by the host after obtaining and expressing the genes for them in the same manner as described above. As used herein, the “antibody” also means to include these antibody fragments.

Modified anti-MIF antibodies including those conjugated with various molecules such as polyethylene glycol (PEG) can also be used. As used herein, the “antibody” also includes these modified antibodies. Such modified antibodies can be obtained by chemically modifying the antibodies obtained as above. Methods for modifying antibodies have already been established in this field of art.

Antibodies used in the present invention may also be bispecific antibodies. Bispecific antibodies may have antigen-binding sites recognizing different epitopes of the MIF molecule or may have one antigen-binding site recognizing MIF and another antigen-binding site recognizing a non-MIF such as a chemotherapeutic agent or a cell-derived toxin. Bispecific antibodies can be prepared by joining HL pairs of two antibodies, or fusing hybridomas producing different monoclonal antibodies to prepare a fusion cell producing a bispecific antibody. Bispecific antibodies can also be prepared by genetic engineering techniques.

The gene for the antibody constructed above can be expressed and obtained by known methods. In mammalian cells, the gene for the antibody can be expressed by operably linking conventional useful promoters, the gene to be expressed and a polyA signal downstream of the 3′ end. For example, promoters/enhancers include human cytomegalovirus immediate early promoters/enhancers.

Other promoters/enhancers that can be used for the antibody expression in the present invention include viral promoters/enhancers derived from retroviruses, polyomaviruses, adenoviruses, simian virus 40 (SV40) or the like or promoters/enhancers derived from mammalian cells such as human elongation factor 1α (HEF1α).

Gene expression can be readily performed by the method of Mulligan et al. (Nature (1979) 277, 108) using SV40 promoters/enhancers or by the method of Mizushima et al. (Nucleic Acids Res. (1990) 18, 5322) using HEF1α promoters/enhancers.

In E. coli, the gene for the antibody can be expressed by operably linking conventional useful promoters, a signal sequence for secreting the antibody and the gene for the antibody to be expressed. Promoters include e.g. lacz promoter and araB promoter. It can be expressed by the method of Ward et al. (Nature (1098) 341, 544-546; FASEB J. (1992) 6, 2422-2427) using lacz promoter or the method of Better et al. (Science (1988) 240, 1041-1043) using araB promoter.

When the antibody is to be produced in periplasms of E. coli, the pelB signal sequence (Lei, S. P. et al., J. Bacteriol. (1987) 169, 4379) can be used as a signal sequence for secreting the antibody. The antibody produced in periplasms is isolated and then used by suitably refolding the structure of the antibody.

Suitable origins of replication include those derived from SV40, polyomaviruses, adenoviruses, bovine papilloma virus (BPV), etc., and expression vectors can contain selectable markers such as the genes for aminoglycoside transferase (APH), thymidine kinase (TK), E. coli xanthineguanine phosphoribosyl transferase (Ecogpt) and dihydrofolate reductase (dhfr) to increase the copy number of the gene in the host cell system.

Any expression system such as eukaryotic or prokaryotic system can be used to prepare antibodies used in the present invention. Suitable eukaryotic cells include animal cells such as established mammalian cell lines, insect cell lines, fungal cell lines and yeast cell lines, and prokaryotic cells include, e.g., bacterial cells such as E. coli cells. Preferably, antibodies used in the present invention are expressed in mammalian cells such as CHO, COS, myeloma, BHK, Vero and HeLa cells.

Then, transformed host cells are cultured in vitro or in vivo to produce a desired antibody. The host cells are cultured by known methods. For example, DMEM, MEM, PRMI1640 and IMDM can be used as culture media optionally in combination with serum supplements such as fetal calf serum (FCS).

Antibodies expressed and produced as above can be isolated from cells or host animals and purified to homogenicity. Isolation and purification of antibodies used in the present invention can be performed on an affinity column. For example, columns using a protein A column include Hyper D, POROS and Sepharose F.F. (Pharmacia). Any other isolation and purification method conventionally used for proteins can be used without limitation. For example, antibodies can be isolated/purified by appropriately selecting and combining chromatography columns other than affinity columns above, filters, ultrafiltration, dialysis, etc. (Antibodies A Laboratory Manual. Ed Harlow, David Lane, Cold Spring Harbor Laboratory, 1988).

In the present invention, recombinant monoclonal antibodies, which are produced by transforming a host with a suitable vector containing an antibody gene cloned from a hybridoma using genetic engineering techniques (e.g. see Vandamme, A. M. et al., Eur. J. Biochem. (1990) 192, 767-775, 1990), can be used.

The antigen-binding activity of antibodies used in the present invention can be determined by known means (Antibodies A Laboratory Manual. Ed Harlow, David Lane, Cold Spring Harbor Laboratory, 1988).

Suitable methods for determining the antigen-binding activity of anti-MIF antibodies used in the present invention include ELISA (Enzyme-Linked Immunosorbent Assay), EIA (Enzyme Immunoassay), RIA (Radioimmunoassay) or Fluorescent Antibody Assay. When an enzyme immunoassay is used, for example, a sample containing an anti-MIF antibody such as the culture supernatant of cells producing the anti-MIF antibody or a purified form of the antibody is added to a plate coated with MIF. The antigen-binding activity can be evaluated by incubating the plate with a secondary antibody labeled with an enzyme such as an alkali phosphatase and washing it and then adding an enzyme substrate such as p-nitrophenyl phosphate and measuring the absorbance.

Therapeutic agents of the present invention are used for the purpose of treating or improving multiple sclerosis. Multiple sclerosis includes C-MS and OpS-MS.

Effective dosages are selected in the range of 0.001 mg to 1000 mg/kg body weight/dose. Alternatively, dosages can be selected at 0.01 to 100000 mg/body, preferably 0.1 to 10000 mg/body, more preferably 0.5 to 1000 mg/body, still more preferably 1 to 100 mg/body per patient. However, therapeutic agents containing an anti-MIF antibody of the present invention are not limited to these dosages.

Therapeutic agents containing an anti-MIF antibody as an active ingredient of the present invention can be routinely formulated (Remington's Pharmaceutical Science, latest edition, Mark Publishing Company, Easton, USA) optionally in combination with pharmaceutically acceptable carriers and additives.

Isotonizing agents that can be used in therapeutic agents of the present invention include polyethylene glycol; and sugars such as dextran, mannitol, sorbitol, inositol, glucose, fructose, lactose, xylose, mannose, maltose, sucrose and raffinose.

Therapeutic agents of the present invention can further contain surfactants. Typical examples of surfactants include:
nonionic surfactants, e.g., sorbitan fatty acid esters such as sorbitan monocaprylate, sorbitan monolaurate, sorbitan monopalmitate; glycerin fatty acid esters such as glycerin monocaprylate, glycerin monomyristate, glycerin monostearate; polyglycerin fatty acid esters such as decaglyceryl monostearate, decaglyceryl distearate, decaglyceryl monolinoleate; polyoxyethylene sorbitan fatty acid esters such as polyoxyethylene sorbitan monolaurate, polyoxyethylene sorbitan monooleate, polyoxyethylene sorbitan monostearate, polyoxyethylene sorbitan monopalmitate, polyoxyethylene sorbitan trioleate, polyoxyethylene sorbitan tristearate; polyoxyethylene sorbitol fatty acid esters such as polyoxyethylene sorbitol tetrastearate, polyoxyethylene sorbitol tetraoleate; polyoxyethylene glycerin fatty acid esters such as polyoxyethylene glyceryl monostearate; polyethylene glycol fatty acid esters such as polyethylene glycol distearate; polyoxyethylene alkyl ethers such as polyoxyethylene lauryl ether; polyoxyethylene polyoxypropylene alkyl ethers such as polyoxyethylene polyoxypropylene glycol ether, polyoxyethylene polyoxypropylene propyl ether, polyoxyethylene polyoxypropylene cetyl ether; polyoxyethylene alkyl phenyl ethers such as polyoxyethylene nonyl phenyl ether; polyoxyethylene hardened castor oils such as polyoxyethylene castor oil, polyoxyethylene hardened castor oil (polyoxyethylene hydrogenated castor oil); polyoxyethylene beeswax derivatives such as polyoxyethylene sorbitol beeswax; polyoxyethylene lanolin derivatives such as polyoxyethylene lanolin; polyoxyethylene fatty acid amides such as polyoxyethylene stearic acid amide having an HLB of 6-18;
anionic surfactants, e.g., alkyl sulfates having a C10-18 alkyl group such as sodium cetyl sulfate, sodium lauryl sulfate, sodium oleyl sulfate; polyoxyethylene alkyl ether sulfates having an average EO mole number of 2-4 and a C10-18 alkyl group such as sodium polyoxyethylene lauryl sulfate; alkyl sulfosuccinic acid ester salts having a C8-18 alkyl group such as sodium laurylsulfosuccinate; and
natural surfactants, e.g., lecithin; glycerophospholipids; sphingophospholipids such as sphingomyelin; sucrose fatty acid esters of C12-18 fatty acids. Formulations of the present invention can contain one or more of these surfactants in combination.

Therapeutic agents of the present invention can further contain diluents, solubilizing agents, excipients, pH-modifiers, soothing agents, buffers, sulfur-containing reducing agents, antioxidants or the like, if desired. For example, sulfur-containing reducing agents include N-acetylcysteine, N-acetylhomocysteine, thioctic acid, thiodiglycol, thioethanolamine, thioglycerol, thiosorbitol, thioglycolic acid and salts thereof, sodium thiosulfate, glutathione, and sulfhydryl-containing compounds such as thioalkanoic acid having 1 to 7 carbon atoms. Antioxidants include erythorbic acid, dibutylhydroxytoluene, butylhydroxyanisole, α-tocopherol, tocopherol acetate, L-ascorbic acid and salts thereof, L-ascorbyl palmitate, L-ascorbyl stearate, sodium bisulfite, sodium sulfite, triamyl gallate, propyl gallate or chelating agents such as disodium ethylenediamine tetraacetate (EDTA), sodium pyrophosphate and sodium metaphosphate. Other components commonly added may also be contained, e.g., inorganic salts such as sodium chloride, potassium chloride, calcium chloride, sodium phosphate, potassium phosphate and sodium bicarbonate; and organic salts such as sodium citrate, potassium citrate and sodium acetate.

Therapeutic agents of the present invention can be prepared by dissolving these components in a buffer such as a phosphate buffer. A preferred pH is 5-8.

Therapeutic agents of the present invention are typically administered via parenteral routes such as injection (e.g. subcutaneous, intravenous, intramuscular or intraperitoneal injection) or percutaneous, mucosal, nasal or pulmonary administration, but may also be orally administered.

Therapeutic agents of the present invention may be in the form of solution formulations or freeze-dried formulations to be dissolved/reconstituted before use. Suitable excipients for freeze-drying include sugar alcohols or sugars such as mannitol or glucose.

The amount of the anti-MIF antibody contained in formulations of the present invention can be determined depending on the type of the disease to be treated, the severity of the disease, the age of the patient and other factors, but a final concentration is generally 0.1-200 mg/ml, preferably 1-120 mg/ml.

In the present invention, MIF expression in the CNS of MS patients was evaluated by in situ hybridization to investigate the potential involvement of MIF in MS. As a result, high MIF mRNA expression in MS lesions was observed, suggesting that MIF might be directly related to disease progression.

To better understand the pathological role of MIF in MS, MIF levels in the cerebrospinal fluid (CSF) of patients with two different types of MS, the conventional form of multiple sclerosis C-MS and the optic-spinal form of the condition OpS-MS, were examined. As a result, high levels of MIF in the CSF of MS patients were observed. Patients with either C-MS or OpS-MS in acute relapse had significantly higher MIF concentrations in the CSF than control subjects. Moreover, MIF levels in the CSF of OpS-MS patients in relapse were higher than those in C-MS patients in relapse. The mean MIF level in the CSF of C-MS patients in relapse was significantly higher than in the remitting phase. On the other hand, the average values of the C-MS group in the remitting phase and the control group were not significantly different. These results showed a correlation between MS and MIF levels.

In the present invention, a model of experimental allergic encephalomyelitis (EAE) was further used to evaluate the role of MIF in the regulation of autoimmune diseases. As a result, an anti-MIF antibody was effective for improvement of the clinical course of MS in a mouse model of EAE.

Also, experimental autoimmune uveoretinitis (EAU) was induced in rats following immunization with a retinal antigen, interphotoreceptor retinoid-binding protein (IRBP). In this experiment, rats were immunized with a single injection of IRBP-derived peptide (ADGSSWEGVGVVPDV) and a neutralizing monoclonal antibody against MIF was injected intraperitoneally every second day from day 0 to day 6, or from day 8 to day 14. T-cell proliferative responses against the peptide were inhibited in rats treated with anti-MIF monoclonal antibodies, and the development of EAU was significantly delayed in rats treated from day 0 to day 6 in comparison with the group treated from day 8 to day 14. These facts indicate that MIF may play an important role in the early phase of EAU, and contribute to the initiation of immune-mediated neurological disorders.

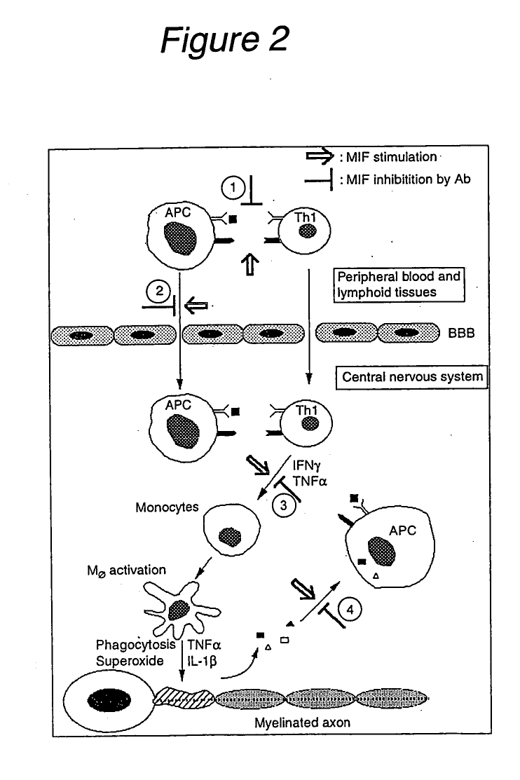

Although the present invention is not bound by any theory, it is believed to be based on the hypothetical mechanism of action of anti-MIF antibody shown in FIG. 2. That is, the anti-MIF antibody may show inhibitory action on multiple sites within the pathophysiology of MS: 1. activation of T-lymphocytes; 2. chemotaxis of monocytes from circulation to the CNS tissue through the blood-brain barrier (BBB); 3. release of inflammatory cytokines (e.g. TNFα, IFNγ) from antigen-presenting cells (APC); 4. phagocytosis of myelin debris by APC.

### INDUSTRIAL APPLICABILITY

Therapeutic agents for multiple sclerosis containing a substance inhibiting the binding of macrophage migration inhibitory factor (MIF) to its receptors as an active ingredient of the present invention were shown to be novel and effective therapeutic agents for multiple sclerosis.

The following examples further illustrate the present invention without, however, limiting the scope of the invention thereto. Various changes and modifications can be made by those skilled in the art on the basis of the description of the invention, and such changes and modifications are also included in the present invention.

### EXAMPLES

### Example 1

### MIF Expression in the CNS of MS Patients

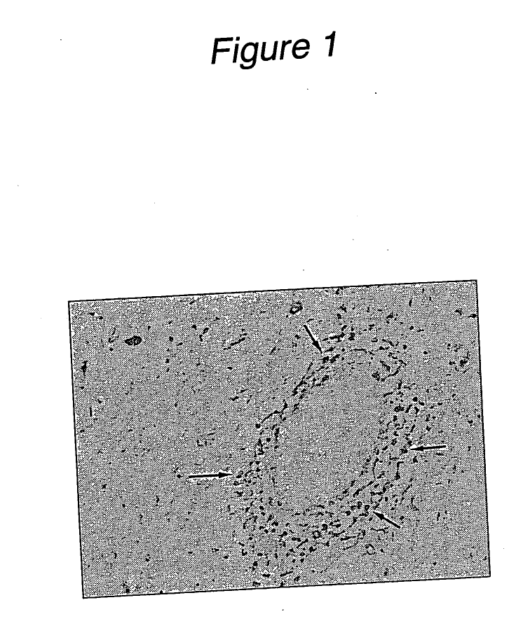

The typical pathological features of MS are perivascular inflammatory cell infiltration and myelin loss, mostly around the periventricular white matter, in the optic nerve tract, brainstem and corpus callosum. During demyelination, resident microglial cells and astrocytes become activated, followed by the development of astrogliosis. An autopsy specimen from a medulla lesion of a 21-year-old woman with MS was observed. It showed demyelination and perivascular leukocyte infiltration demonstrating typical active MS plaques. In situ hybridization revealed that MIF mRNA was expressed in perivascular leukocytes, astrocytes and microglia in the white matter lesion (FIG. 1). High MIF mRNA expression in MS lesions suggests that MIF might be directly related to the disease progression.

### Example 2

### MIF Levels in the CNS of MS Patients

To better understand the pathological role of MIF in MS, MIF levels in the cerebrospinal fluid (CSF) of patients with two different types of MS, the conventional form of multiple sclerosis (C-MS) and the optic-spinal form of the condition (OpS-MS), were examined.

The results are shown in Table 1 below.

TABLE 1
MIF levels in CSF

| | MIF (ng/ml) |
|---|---|
| Control | 2.38 ± 0.18 |
| C-MS in relapse | 4.13 ± 0.30 |
| in remission | 2.65 ± 0.19 |
| OpS-MS in relapse | 5.53 ± 0.66 |

Note:
Values are expressed in mean ± SD (n = 5).

High levels of MIF in the CSF of patients with the immune-mediated diseases were observed. Patients with either C-MS or OpS-MS in acute relapse had significantly higher MIF levels in the CSF than control subjects. Moreover, MIF levels in CSF of OpS-MS patients in relapse were higher than those in C-MS patients in relapse. The mean MIF level in the CSF of C-MS patients in relapse was significantly higher than in the remitting phase. On the other hand, the average values of the C-MS group in the remitting phase and the control group were not significantly different. When the mean MIF levels in CSF were compared between C-MS patients in remission and OpS-MS patients, the mean MIF level of the OpS-MS patients was significantly higher than that of the C-MS patients. Thus, it is possible that the higher MIF levels in CSF of OpS-MS than C-MS patients may be associated with damaged endothelial cells in the CNS.

### Example 3

### Effects of Anti-MIF Antibodies on an EAE Mouse Model

Investigation of the T-cell receptor and encephalitogenic epitopes involved in EAE has led to a number of experimental therapeutic strategies that have potential relevance to the treatment of MS. It is known that chronic relapsing EAE is an autoimmune disorder characterized by inflammation and demyelination in the CNS. In brief, the adoptive transfer of myelin basic protein (MBP)-specific CD4+ class II MHC-restricted T-cells into naive syngeneic mice produces pathological states resembling MS in humans (Pettinelli CB et al., J Immunol (1981) 127:1420-1423). Accordingly, a model of EAE was used to evaluate the role of MIF in the regulation of autoimmune diseases.

<Scoring of Pathological Tissues>

EAE was induced in C57 mice by the known method described above (Pettinelli CB et al., J Immunol (1981) 127:1420-1423). The mice in which EAE was induced were treated with 200 μg of an anti-MIF antibody (a polyclonal antibody prepared in-house by immunizing rabbits with mouse recombinant MIF) three times at intervals of 48 hours (started 24 hours after EAE induction).

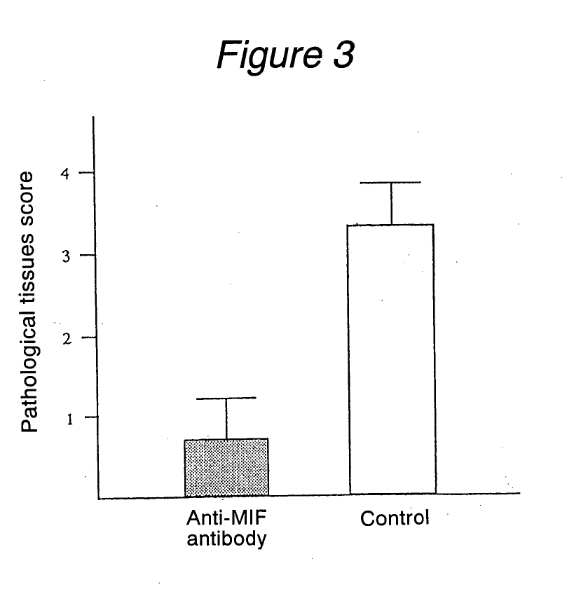

The mice treated with the anti-MIF antibody 24 hours after EAE induction (n=20) and control mice not treated with the anti-MIF antibody (a control group treated with rabbit non-immune IgG, n=20) were scored for pathological tissues on the basis of the degree of cellular infiltration or tissue destruction or the like (as expressed in averages in the mice tested). The scoring results of pathological tissues are shown in FIG. 3. Improvements in the scores of pathological tissues were observed in the mice treated with the anti-MIF antibody.

<Scoring of Clinical Symptoms>

EAE was induced in C57 mice by the known method described above (Pettinelli C B et al., J Immunol (1981) 127:1420-1423). The mice in which EAE was induced were treated with 200 μg of an anti-MIF antibody (a polyclonal antibody prepared in-house by immunizing rabbits with mouse recombinant MIF) three times at intervals of 48 hours (started 24 hours after EAE induction).

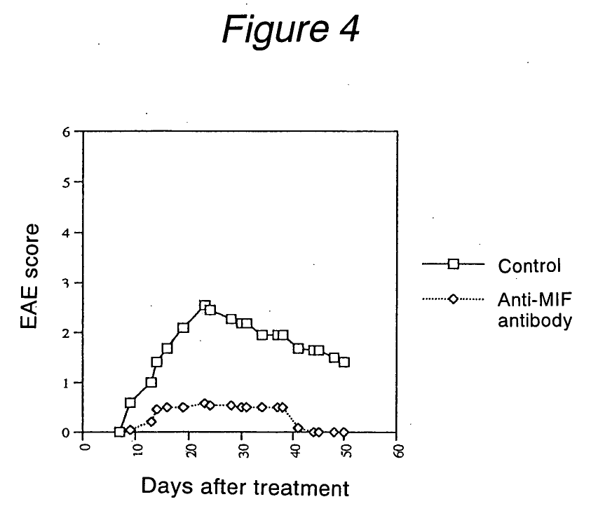

The mice treated with the anti-MIF antibody (n=20) and control mice not treated with the anti-MIF antibody (a control group treated with rabbit non-immune IgG, n=20) were observed and scored for clinical symptoms from day 1 to 50 after EAE induction (as expressed in averages in the mice tested). The clinical symptoms were scored on the basis of common clinical symptoms of EAE (e.g. gait difficulty). The scoring results of clinical symptom are shown in FIG. 4. Improvements in the scores of clinical symptoms were observed in the mice treated with the anti-MIF antibody.

These results showed that the anti-MIF antibody is effective for improving the clinical course of MS in the mouse model of EAE.

1. A therapeutic agent for multiple sclerosis containing an antibody binding to macrophage migration inhibitory factor (MIF) as an active ingredient.

2. A therapeutic agent for multiple sclerosis containing an antibody binding to macrophage migration inhibitory factor (MIF) to inhibit the binding of macrophage migration inhibitory factor (MIF) to its receptors as an active ingredient.

3. The therapeutic agent for multiple sclerosis of claim 1 wherein multiple sclerosis is the conventional form of multiple sclerosis (C-MS) or the optic-spinal form of multiple sclerosis (OpS-Ms).

4. The therapeutic agent for multiple sclerosis of claim 2 wherein multiple sclerosis is the conventional form of multiple sclerosis (C-MS) or the optic-spinal form of multiple sclerosis (OpS-Ms).


SEQUENCE LISTING (1 sequence)

SEQ ID NO: 1 - protein, 15 aa, Artificial Sequence
  Description of Artificial Sequence Synthetic peptide
ADGSSWEGVGVVPDV